(19) United States
(12) Patent Application Publication
(10) Pub. No.: US 2023/0066065 A1
(43) Pub. Date: Mar. 2, 2023

(54) USE OF LOW DOSES OF HYDROXYCHLOROQUINE FOR THE TREATMENT OF LIPIN-1 DEFICIENCY

(71) Applicants: Institut National de la Santé et de la Recherche Médicale (INSERM), Paris (FR); Assistance Publique-Hôpitaux de Paris (APHP), Paris (FR); Centre National de la Recherche Scientifque (CNRS), Paris (FR); Fondation Imagine, Paris (FR); Université Paris Cité, Paris (FR)

(72) Inventors: Pascale DE LONLAY-DEBENEY, Paris (FR); Peter VAN ENDERT, Paris (FR); Sebastian MONTEALEGRE, Paris (FR); Caroline TUCHMANN-DURAND, Paris (FR); Laure CACCAVELLI, Paris Cedex 14 (FR); Marine MADRANGE, Paris Cedex 14 (FR); Perrine RENARD, Paris (FR)

(21) Appl. No.: 17/797,265

(22) PCT Filed: Feb. 4, 2021
(86) PCT No.: PCT/EP2021/052668

§ 371 Date: Aug. 3, 2022

(30) Foreign Application Priority Data
Feb. 5, 2020 (EP) 20305104.0

(51) Int. Cl. A61K 31/4706; A61P 3/00

(52) U.S. Cl. CPC A61K 31/4706; A61P 3/00

(57) ABSTRACT

Lipin-1 deficiency is a rare, life-threatening condition that causes severe rhabdomyolysis episodes (RM) triggered by febrile illness and effort. Now, the inventors treated 10 patients with LPIN1 mutations with hydroxychloroquine (HCQ) in an off open-label use phases 1 and 2 study, to assess safety, clinical, and biological effects of the drug. A first inclusion group of patients were treated with oral HCQ at a dose of 6.5 mg/Kg/day in one intake, not exceeding 400 mg/day. Five patients have not presented any new acute RM under treatment, except for 2 patients experimented one and two episodes of RM respectively despite HCQ in a context of gastroenteritis. Plasma levels of HCQ were in the range of 400 ng/ml except in the two patients who experimented RM, in whom the plasma HCQ levels were higher (1000 ng/ml). With a therapeutic adjustment, in order to maintain plasma levels of HCQ under 700 ng/ml, in a new group of patients, two patients did not suffer from new acute RM under treatment. HCQ had not seem to have benefit effect for one patient. Thus, the inventors describe the first human experience with HCQ for Lipin-1 disease. The results allow the inventors to propose low doses of HCQ as a long-term treatment to prevent further relapses in this severe disease.

### FIELD OF THE INVENTION

The present invention is in the field of medicine.

### BACKGROUND OF THE INVENTION

Lipin-1 deficiency is an inherited autosomal recessive disease due to mutations in the LPIN1 gene. Homozygous or compound heterozygous LPIN1 mutations causes acute and recurrent episodes of rhabdomyolysis (RM) and myoglobinuria in children triggered by febrile illness, fasting or exercise¹⁻⁵. The onset of the disease is usually early, as the first episodes of RM occur before age 6 years. Their number in infancy ranges from 1 to several dozens per patient. Their characteristic is to be very severe (CK>10,000 UI/L, mostly >50,000 UI/L), making this disease a life-threatening condition in the absence of curative treatment⁴. The period of high risk for the Lipin-1 disease is during infancy when the risk of viral illness is more important than in adult age. However, the risk of RM remains all the life⁶. Mortality rate is around 10%, during myoglobinuric bouts. The poor prognosis results in cardiac arrhythmia during the first hours of RM despite the use of aggressive management in intensive care units, due to hyperkalemia^{1-5,7-9}, majored with renal failure, and possibly potentiated by specific cardiac involvement^{10,11}. Indeed, several patients presented cardiomegaly at the time of their death, and one of the inventor's patients suffers from cardiac failure independently of rhabdomyolysis bouts¹¹. When they survive, CK levels normalize or are subnormal between the episodes of RM until the next episode. However, some patients can have permanent (chronic) mild elevation of CK (about 1000 UI/L). Between RM episodes, patients have a normal physical examination and do not present any symptoms, or present permanent muscle pain weakness and/or cardiomyopathy^{3,6,11}. A specific treatment does not exist. Altogether, these observations indicate that there is a crucial need to identify a treatment.

Lipin-1, which is abundantly expressed in adipocytes and skeletal muscle¹², plays a dual role¹³ i) as a phosphatidate acid phosphatase (phosphatidic acid phosphatase 1, PAP1, EC 3.1.3.4) that dephosphorylates phosphatidic acid to diacylglycerol (DAG) and contributes to triacylglycerol and phospholipid biosynthesis¹⁴⁻¹⁶ and ii) as a transcriptional co-activator associating with PPARα, SREBP1 and PGC-1α that regulate the expression of genes encoding proteins involved in mitochondrial fatty acid oxidation and energetic pathways¹⁷. PPARα, SREBP1 and PGC-1α expression patterns were normal in adipocytes¹⁸ and skeletal muscle¹⁹ from Lipin-1 deficient patients.

Because Lipin-1-related RM are precipitated by febrile illness and effort, conditions that are associated with high circulating levels of pro-inflammatory mediators, we suspected a major role of inflammation. The inventors pioneer observation that a high level of pro-inflammatory cytokines can be detected in patient sera, especially during flares led them to test whether or not this may be explained by a preferential activation of a Pattern-Recognition Receptor (PRR), such as Toll-like Receptors (TLR)²⁰ (in review). Exposing LPIN1 deficient myoblasts and dendritic cells to various TLR agonists in vitro revealed a hypersensitivity of patient cells specifically to agonists of TLR9, an endosomal TLR that requires an activating cleavage by endolysosomal proteases. This intriguing phenotype was recapitulated by inactivating control cells for LPIN1. Finally, the inventors identified the pathogenic sequence linking Lipin-1 deficiency and RM (in review). The loss of enzymatic activity of Lipin-1 leads to reduction of the phosphatidylinositol 3-kinase (PKD)-Vps34 and phosphatidylinositol 3-phosphate (PI3P) specifically at the membrane of late endosomes in patients cells. In animal models, reduction of PKD-Vps34 and PI3P levels induces a blockade in autophagic flux²¹⁻²³. Importantly, phosphoinnositides are key modulators of autophagy²⁴, notably by the regulation of Ras-related proteins in brain (Rab proteins) that are involved in cell trafficking and are dependent of their immediate lipid microenvironment^{25,26}. In human myoblasts, the inventors confirmed the reduction of PKD-Vps34 and PI3P levels and discovered the pathogenic sequence that results in: i) a reduction of the binding of the FYCO1 protein to the small GTPase Rab7; ii) the accumulation of enlarged late endosome structures at the perinuclear region associated to a higher binding of the GTP-bound Rab7 form to the effector protein RILP; iii) a perturbation of late endosomal architecture and functions including a defect in lysosomal degradation, in autophagic clearance and mitophagic elongation in patient cells.

Mitochondrial quality control is critical for maintaining mitochondrial homeostasis²⁷, particularly in postmitotic tissues such as skeletal muscle^{28,29}. When damaged, or in response to many different stimuli including cytokines and environmental stresses, mitochondria generate stress signals resulting in the loss of mitochondrial membrane potential, with the disruption of ATP synthesis and the release of mtDNA and ROS^{30,31}. They promote mitophagy³², a selective form of autophagy that specifically targets mitochondria through autophagolysosomal activity in response to the stresses^{33,34} for degradation, before activation of cell death³⁵. Thus mitophagy is the key mechanism preventing the release of highly immune-stimulatory mtDNA³⁶, notably via activation of TLR9 pathway³⁷⁻⁴⁰. As a consequence of a defective autophagy and mitophagy, RM could be reproduced in Lipin-1-deficient myoblasts exposed to TLR9 agonist (CpG-A) and to a protein-free minimal medium (EBSS medium), the combination of both mimicking a febrile infection, with an accumulation of oxydized DNA (oxDNA) in late endosomal/lysosomal structures. The inventors suspect that the main part of oxDNA corresponds with mtDNA, being a well-documented natural ligand of TLR9⁴⁰ which in turn increases secretion of inflammatory cytokines. The consequences are the release of calcium into cytoplasm and the cell death, two hallmarks of RM⁴¹. Silencing LPIN1 in control myoblasts reproduces the alterations in the cell. On the other hand, a TLR9 antagonist prevents both calcium release and cell death induced by TRL9 activation, suggesting novel therapeutic perspectives for Lipin-1 disease. To note, intensive exercise also triggers RM, probably because it releases cytokines, DNA damage and oxidative stress from skeletal muscle^{42,43}.

Hydroxychloroquine sulfate (HCQ) and Chloroquine (CQ), belonging to the pharmacological classification of 4-aminoquinolones, are well known to have TLR9 antagonist proprieties, although their mechanisms of actions remain still unclear and probably depend on the cell types⁴⁴; ⁴⁵. They are essential therapies in patients with immune-mediated inflammatory disorders such as systemic lupus erythematosus (SLE), rheumatoid arthritis, and Sjogren's syndrome^{46 47} by their anti-inflammatory, immune-suppressive and photoprotective effects. Similarly to CpG-A, pretreating Lipin-1 deficient myoblasts exposed to TLR9 antagonist and stress conditions restored calcium homeostasis and prevented cell death (YH). Accordingly, treatment of rhabdomyolysis with TLR9 antagonists was thus suggested (WO2017085115).

### SUMMARY OF THE INVENTION

As defined by the claims, the present invention relates to methods of the treatment of Lipin-1 deficiency.

### DETAILED DESCRIPTION OF THE INVENTION

Lipin-1 deficiency is a rare, life-threatening condition that causes severe rhabdomyolysis episodes (RM) triggered by febrile illness and effort. Chronic cardio muscular symptoms are occasionally associated. Mortality is important despite aggressive management. The inventors recently showed that Lipin-1-deficient myoblasts exhibit perturbations in lysosomal degradation and mitophagic elongation. The consequences in the myoblasts exposed to Toll-Like Receptor-9 (TLR9) ligand and to a protein-free minimal medium, a setting mimicking febrile illness, are an accumulation of mitochondrial DNA (mtDNA), a hyperactivation of TLR9, an increase in inflammatory cytokine production, an aberrant calcium release into the cytosol and an increase of cell death. Pretreating myoblasts with a TLR9 antagonist corrects in vitro the phenotype.

Based on the first in vitro results and in absence of animal models, the inventors treated patients with LPIN1 mutations with HCQ in an off open-label use phases 1 and 2 study, to assess safety, clinical, and biological effects of the drug. The primary objectives consisted in the evaluation of the clinical tolerance of HCQ, as well as the clinical and biological outcomes after initiation of HCQ treatment. The secondary objective was to characterize the mechanism of action of HCQ in primary myoblasts, notably dose-dependent effects and their relationship with autophagy upon cellular stress. Ten patients were treated by HCQ in this off open-label use, treated at the beginning with oral HCQ at a dose of 6.5 mg/Kg/day in one intake, not exceeding 400 mg/day.

Clinical and biological parameters were evaluated in parallel of the dosage of HCQ, performed on total blood samples with HPLC. Biological parameter consisted in measuring pro-inflammatory cytokines in plasma with ELISA. Physical condition included muscle pain (VAS score), quality-of-life assessment (PedsQL™ score), the distance walked during the 6-minute test, the number of steps climbed during the 3-minute test, cardiac function and sub-maximal exercise test. In vitro studies were performed on primary myoblasts from 4 patients and healthy controls. Autophagy was analyzed by measuring the ratio between LC3-II and B-actin by Western Blot. Oxidized DNA was studied by immunofluorescence staining. MtDNA levels was performed by using qPCR.

In the all patients studied, the inventors observed a reduction in plasma inflammatory cytokines, and a general improvement of physical capacities.

A first inclusion group of patients were treated with oral HCQ at a dose of 6.5 mg/Kg/day in one intake, not exceeding 400 mg/day. Five patients have not presented any new acute RM under treatment, except for 2 patients experimented one and two episodes of RM respectively despite HCQ in a context of gastroenteritis. Plasma levels of HCQ were in the range of 400 ng/ml except in the two patients who experimented RM, in whom the plasma HCQ levels were higher (1000 ng/ml). With a therapeutic adjustment, in order to maintain plasma levels of HCQ under 700 ng/ml, in a new group of patients, two patients did not suffer from new acute RM under treatment. HCQ had not benefit effect in one patient.

In vitro studies showed that HCQ potentiated the autophagy blockage induced by the immunometabolic stress both in control and patients myoblasts. This deleterious effect was dose dependant: the LC3-II accumulation was more important at high (10 μM) HCQ concentration than at low (0.1 and 1 μM) HCQ concentrations. By contrast, HCQ presented beneficial effects by preventing oxidized DNA and mtDNA accumulation during immunometabolic stress condition.

Thus, the inventors describe the first human experience with HCQ for Lipin-1 disease. The observed beneficial effects in patients are consistent with findings in myoblasts, with a dose dependent effect: deleterious effect on autophagy at high dose, and beneficial effect on oxDNA without negative impact on autophagy at low dose. The results allow the inventors to propose low doses of HCQ as a long-term treatment to prevent further relapses in this severe disease.

Accordingly, the first object of the present invention relates to a method of treating a patient suffering from Lipin-1 deficiency, the method comprising administering an amount of hydroxychloroquine sufficient to elicit a plasma level that does not exceeds 700 ng/ml.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “hydroxychloroquine” or “HCQ” has its general meaning in the art and refers to 2-[[4-[(7-Chloro-4-quinolyl) amino] pentyl] ethylamino] ethanol sulfate (1:1). Methods of synthesis for hydroxychloroquine are disclosed in U.S. Pat. No. 2,546,658, herein incorporated by reference. In some embodiments, the patient is administered with hydroxychloroquine sulfate (Plaquenil®).

As used herein, the term “plasma level” or “plasma concentration” refers to the amount of HCQ present in the plasma of a treated patient. Typically, the plasma level is determined by any method well known in the art (see e.g. EXAMPLE).

In some embodiments, HCQ is administered to the patient so as to elicit a plasma level that ranges from 100 ng/ml to 700 ng/ml. In some embodiments, HCQ is administered to the patient so as to elicit a plasma level of about 100; 150; 200; 250; 300; 350; 400; 450; 500; 550; 600; 650; or 700 ng/ml.

As used herein, the term “about,” as applied to one or more values of interest, refers to a value that is similar to a stated reference value. In some embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

Typically, HCQ may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intra-peritoneal, intramuscular, intravenous, sub-dermal, transdermal, intrathecal and intranasal administration forms and rectal administration forms. Preferably, the pharmaceutical compositions contain vehicles, which are pharmaceutically acceptable for a formulation capable of being injected. These may be in particular isotonic, sterile, saline solutions (monosodium or disodium phosphate, sodium, calcium or magnesium chloride and the like or mixtures of such salts), or dry, especially freeze-dried compositions which, upon addition, depending on the case, of sterilized water or physiological saline, permit the constitution of injectable solutions.

The invention will be further illustrated by the following figures and examples. However, these examples and figures should not be interpreted in any way as limiting the scope of the present invention.

### FIGURES

FIG. 1.(A-D) Clinical evaluation prior and following treatment with HCQ. (A) Pain Evaluation (VAS score); B: Quality-of-life assessment (PedsQL™ score) adapted to age for children and parents; (C): The 6-minute walk distance (6 MWT); D: The 3-minute step test (3 MST).

FIG. 2.(A-B) Exercise tolerance and cardiac function evaluated by the dQ/dVO2 rate prior, following and after discontinuation of HCQ treatment; cycle ergometer.

FIG. 3.(A-B) Inflammatory cytokine levels in plasma prior to and following the treatment with HCQ; ELISA.

FIG. 4.(A-F) Autophagy evaluated by the quantity of LC3-II in Lipin-1 deficient and control myoblasts; comparison between basal condition and immunometabolic stress (A-B) and after HCQ treatment compared to immunometabolic stress (C-D) or basal condition (E-F); Immunoblot. GM: growth medium; E: EBSS; C: CpG-A; HCQ: Hydroxychloroquine.

FIG. 5.(A-C) Assessment of 8-OHdG in Lipin-1 deficient and control myoblasts; comparison between basal condition and immunometabolic stress (A-B) and after HCQ treatment compared to immunometabolic stress (C); Immunofluorescence. GM: growth medium; E: EBSS; C: CpG-A; HCQ: Hydroxychloroquine.

FIG. 6.(A-B) Evaluation of mtDNA in Lipin-1 deficient myoblasts; comparison between basal condition and immunometabolic stress (A) and after HCQ treatment compared to immunometabolic stress (B); qPCR. GM: growth medium; E: EBSS; C: CpG-A; HCQ: Hydroxychloroquine.

FIG. 7.(A-C) Relative TLR9 expression in Lipin-1 deficient and control myoblasts; comparison between basal condition and immunometabolic stress (A-B) and after HCQ treatment compared to immunometabolic stress (C); RT-qPCR. compared to basal condition in myoblasts. GM: growth medium; E: EBSS; C: CpG-A; HCQ: Hydroxychloroquine.

### EXAMPLE

Methods

Off Open-Label Use Phases 1 and 2 Study

Ten patients with Lipin-1 deficiency were progressively enrolled in an off open-label use phases 1 and 2 study to assess safety, clinical, and biological effects of Hydroxychloroquine Sulfate (HCQ). The diagnosis of Lipin-1 disease was confirmed in all patients (Table 1). LPIN1 genotyping was performed by PCR analysis of DNA isolated from blood as previously described². The patients presented severe episodes of acute RM (1 to 6) defined as creatine kinase (CK) levels >6 000 U/L (normal<160 U/L), associated with mild viral illness, fasting, or prolonged exercise. Chronic symptoms such as muscle pain, fatigability, exercise intolerance, or more rarely cardiac feature were associated in 6 patients.

The patients were treated with oral HCQ at an initial posology of 6.5 mg/Kg/day in one intake, not exceeding 400 mg/day, with different treatment duration depending on the patient (Table 2). Tablets (200 mg) were proposed to patients over 6 years old, and soluble form to patients under 6 years old.

Efficacy and safety of HCQ in patients were assessed by clinical and biological evaluation determined at baseline before HCQ treatment and at regular intervals after initiation of HCQ treatment (every 3 to 6 months).

The clinical parameters encompassed the RM episode number and several standardized tools based on age at study start: auto evaluations assessing the muscle pain and the quality of life, 6-Minute Walk Test, 3-Minute Step Test, heart ultrasounds, and exercise tolerance test in the subgroup of subjects>6 years old¹¹.

VAS questionnaire was completed by the patient and his parents for evaluating muscle pain, ranged from score 4: not painful, to 40: very painful. Two Quality-of-life assessment (PedsQL™) adapted to the age of the patient were completed by either the patient or his parents. The score ranged from 0: very good quality of life, to 92: very bad quality of life.

Muscle function was assessed by walking capacity within a 6-Minute Walk Test (6 MWT)^{48,49} and by stair climbing capacity within a 3-Minute Step Test (3 MST)⁵⁰. The objectives were to walk and climb steps as far as possible for 6 and 3 minutes respectively.

Echocardiography was performed using a Vivid 9 system (General Electric Vingmed Ultrasound, Horten, Norway). Transducers (12 to 4 Hz) were chosen based on patient size and morphology to obtain the best image resolution. Images were acquired and stored in a digital format by two experienced examiners for offline analysis using EchoPac version 112.0.1 software (General Electric Vingmed). All records were performed while the patient was in sinus rhythm under continuous monitoring via electrocardiogram. All measurements of ventricular dimensions and function were performed according to the guidelines of the American and European Societies of Echocardiography⁵¹.

The global longitudinal strain of the left ventricle was defined as the percentage of change during myocardial deformation and was evaluated through the speckle-tracking method using EchoPac (2D strain Q analysis) version 112.0.1 (General Electric Vingmed)¹¹.

Patients≥6 years old underwent cardiopulmonary exercise tests (CPETs). CPETs were performed to assess the maximal exercise capacity using a cycle ergometer¹¹. The CPET data were compared with retrospectively collected data from a population of 21 paired children who required non-invasive cardiac output monitoring because of dyspnoea during exercise or chest pain and were considered healthy at the end of the examinations. Pairing was conducted according to sex and age. Oxygen uptake (VO₂) and carbon dioxide output were measured breath-by-breath by an automated system (Sensor Medic, MSE). Measurements were taken at rest and every 20 s throughout exercise and recovery. The exercise test was considered maximal if the patient achieved a respiratory exchange ratio (RER)>1.1 and/or a maximal heart rate>85% of the theoretical maximal heart rate, and/or the oxygen uptake reached a plateau¹¹. Cardiac output (Q) and stroke volume (SV) were determined noninvasively during the exercise test using a thoracic bioelectrical impedance device (PhysioFlow, PF-05 Lab 1, Manatec Biomedical) as routinely performed in our centre. We calculated the slope of the relationship between cardiac output and oxygen utilization (dQ/dVO2) using measurements of Q and VO2 at rest as well as during submaximal and maximal exercise. Normally, cardiac output increases by 5-6 litres per utilized litre of supplementary oxygen (dQ/dVO2 at 5) even when the subject is deconditioned. In contrast, high dQ/dVO2 (>>5) is observed when the oxidative phosphorylation of muscle is impaired^{52 11}. Muscle oxygenation was measured using a near-infrared spectroscopy (NIRS) device (SenSmart X-100, NONIN) in the patients during CPET.

The biological parameters measured in blood or plasma included HCQ and inflammatory cytokines levels.

The dosage of Hydroxychloroquine was performed on total blood samples at a rate of 5 ml per assay tube, using EDTA tubes, and stored at +4° C. (dosages performed in the pharmacology/toxicology Department of the Hospital Cochin and Hospital La Pitié-Salpétrière). The evaluation of the tolerance of HCQ included interrogatory for allergy and abdominal pain, eye fundus and retinogram at 0, 12, 24 months after HCQ introduction.

Cytokine concentrations were measured in plasma using a ten-plex Cytometric Bead Assay (CBA) kit from BD Biosciences, according to the manufacturer's instructions, then analyzed by flow cytometry (ARIA II; BD Biosciences) with CBA Analysis Software (FCAP Array version 3.0; Soft Flow, St Louis Park, Minn.). The results were expressed in picograms per milliliter.

In Vitro Mechanisms of Action of HCQ

In vitro studies were performed on primary myoblasts from 4 patients (P1, P4, P6, Table 1, and another patient untreated P11) and from healthy controls. Skeletal muscle biopsies were taken from the brachial region (deltoid region) when the patients did not suffer of acute RM for at least two months. The biological collection (plasma, myoblasts) has been declared to the Ministère de la Santé. The study of blood and myoblasts has been approved by the Comité pour la protection des personnes (CPP) of the University Paris XI.

Human primary myoblasts were isolated and grown as described¹⁹. Briefly, CD56+ myoblasts isolated by flow cytometry cell sorting were maintained in HamF10 medium supplemented with 20% fetal calf serum on culture plates coated with 1% gelatin. To mimic RM, F10 medium was replaced by EBSS (minimal essential medium) (4 h) with TLR9 agonist CpGA (ODN2216) at the concentration of 10 μg/ml. Myoblasts were incubated with various HCQ concentrations (0.1 to 10 μM) 18 h before and during myoblasts starvation.

Imuunofluorescence staining. Oxidized DNA on fixed cells was detected with mouse-anti-8-OHdG (sc-66036, Santa Cruz Biotechnology, IF: 1:100)⁵⁴ and analyzed by confocal fluorescence microscopy. Images were recorded on a confocal LSM 700 microscope (Zeiss), with a 63× oil immersion objective and quantification was performed by analyzing cells on the Icy software (v 1.9.5.1 BioImage Analysis unit, Institut Pasteur, France).

MtDNA levels was performed by using qPCR for the mitochondrial12S gene reported to the nuclear β-actin gene. Quantitative PCR was performed in triplicate using the Light Cycler VIIA7 System (Roche).

Autophagy measurements. Autophagy was analyzed using the LC3-II/B-actin ratio by Western Blot. The following antibodies used in this study were: mouse-anti-β-actin (sc-81178, Santa Cruz Biotechnology, western blotting (WB): 1:5000), mouse-anti-LC3B (clone 4E12, MBL International, IF: 1:100).

Statistical analysis. Statistical analysis was performed with GraphPad Prism software using a two-way ANOVA test.

Results

Off Open-Label Use Phases 1 and 2 Study: Safety and Clinical and Biological Effects of HCQ in Patients with Lipin-1 Disease

A total of 10 patients with Lipin-1 disease, 9 children and 1 adult, born from 6 distinct families, were enrolled and treated with Hydroxychloroquine Sulfate (HCQ) in a compassional study (Table 1).

The patients were treated with oral HCQ at a initial posology of 6.5 mg/Kg/day in one intake, not exceeding 400 mg/day, with different treatment duration depending on the patient (Table 2). The data were collected at the Necker Hospital (Reference Center of Metabolic diseases) during the visits: M0 (before treatment), M3 (3 months treatment), M6 then every 6 months. The efficacy outcome of the treatment was measured by clinical and biological endpoints.

HCQ Concentration in Plasma

Levels of HCQ measured at each visit were in the range of 400 ng/ml except in P5 and P6 in whom the plasma HCQ levels were higher (1000 ng/ml) (Table 2). Comparing patients by HCQ concentration, the two patients who experimented RM at the time of gastroenteritis (P5 and P6) were overdosed compared to others. Because of possible deleterious effect of HCQ (autophagic flux blockage, see below), we decided to continue HCQ treatment with lower doses and a strict monitoring (closer) of HCQ in plasma, in order to obtain a therapeutic window, i.e. between 100 and 700 ng/m and avoid an overdosis (>700 ng/ml). This dose adjustment had allowed to not observe new acute RM in the first patients included and in the second wave of patients' inclusion.

Rhabdomyolysis

A summary of the number of RM is shown in Table 2. The numbers of RM prior to the treatment were 29 for all the 10 patients studied.

Seven patients have not presented any new acute RM under treatment and 2 patients (P5 and P6) experimented one and two episodes of RM respectively despite HCQ in a context of gastroenteritis in each case (Table 2). HCQ had not seem to have benefit effect for one patient (P7). It is worth remembering that both patients made severe RM prior to the HCQ treatment and that P6 lost one sibling at age 6 y from a RM in an intensive care unit in 2015, although the diagnosis of Lipin-1 disease was known (personal data). Moreover, the first patient treated (P1) discontinued HCQ during the treatment period (M9), neither for reasons related to side effects of the medication but because of lack of compliance. Among 6 weeks after the HCQ disruption, he underwent extensive RM. No other RM was observed after resumption of treatment.

Clinical Physical Performances

Five patients also presented chronic muscle symptoms (Table 1) including muscle pain, fatigability or exercise intolerance. One patient (P3) developed cardiac failure at age 17 y¹¹. We observed a general improvement of physical condition for all patients tested. For the VAS and PedsQL™ score, P2, P5 and P8 were excluded because of Crohn disease, psychosis and mental retardation respectively. There was no evaluation of exercise test for P8 and few data for P7 did not allow to explore these parameters.

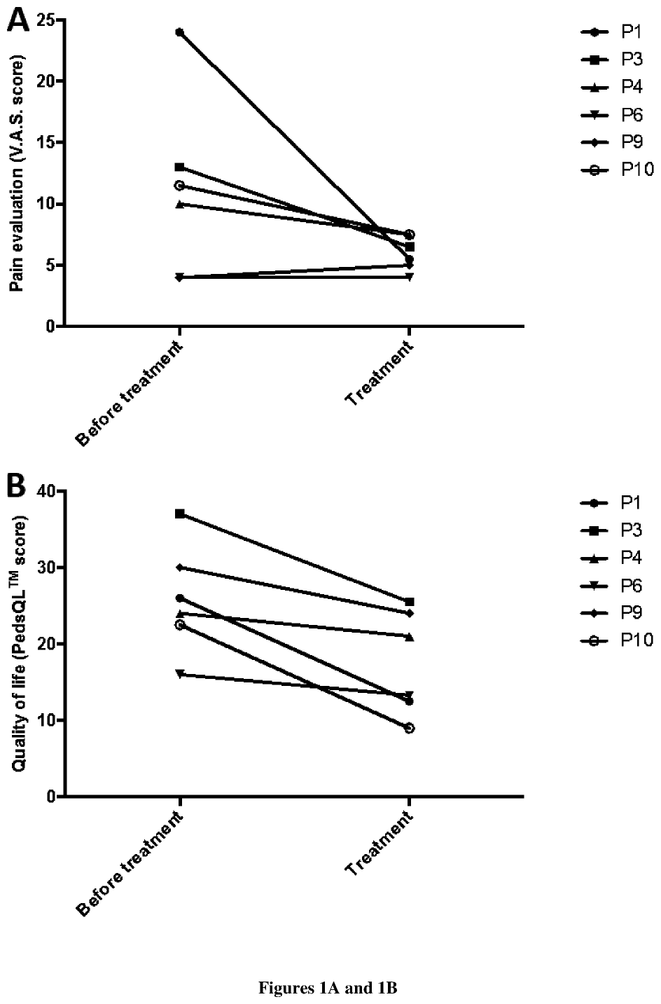

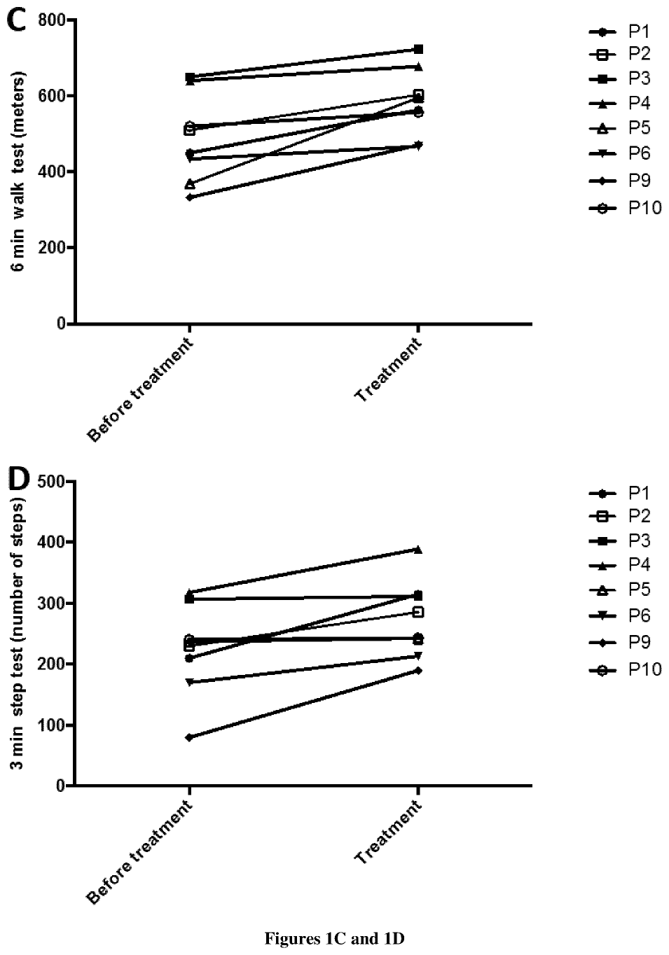

The Assessment of pain using VAS questionnaire (score 4: not painful-40: very painful) completed by the patients and their parents showed a dramatic improvement in 4 patients tested (median score :12.25 prior treatment by HCQ; 7 following treatment) and no difference in 2 other patients (FIG. 1A).

Before treatment, quality-of-life assessment (PedsQL™) adapted to age (child and parent questionnaires revealed a score at 25 in the 6 patients. These patients considered that they had a poor quality of life. Under treatment, the quality of life perceived by the child and the parents was improved with median score at 17.1 (FIG. 1B).

The efficacy outcome of the treatment was also measured by the distance walked during the 6-minute test and by the number of steps climbed during the 3-minute test respectively, for the patients older than 6 y. The distance performed in 6 min (6 MWT, FIG. 1C) and the number of steps ascended in 3 min (3 MST, FIG. 1D) increased, indicating a better physical performance following HCQ treatment, compared to norms published in previous publications⁵⁵⁻⁵⁸. Indeed, median score increased of near 100 meters in overall 6 MWT following treatment compared to results obtained prior treatment by HCQ. Moreover, median score increased of 31 steps in overall 3 MST following treatment compared to results obtained prior treatment by HCQ.

All 9 patients but one displayed normal resting cardiac function before treatment, as determined by echocardiography. P3 exhibited slight left ventricular dysfunction at rest (LVEF at 50% then 45%) and a lack of increased stroke volume during cycle ramp exercise¹¹. For this reason, HCQ was introduced in this patient. He was ameliorated under treatment as his LVEF improved from 45% to 62% at M3, then remained stable at 63% at M6, M9, M12 following treatment (not shown). His fatigability improved in parallel. All other patients conserved normal heart function. His stopped his treatment, and to date, his cardiac dysfunction is deteriorated with a LVEF at 55%. The treatment will be re-administrated.

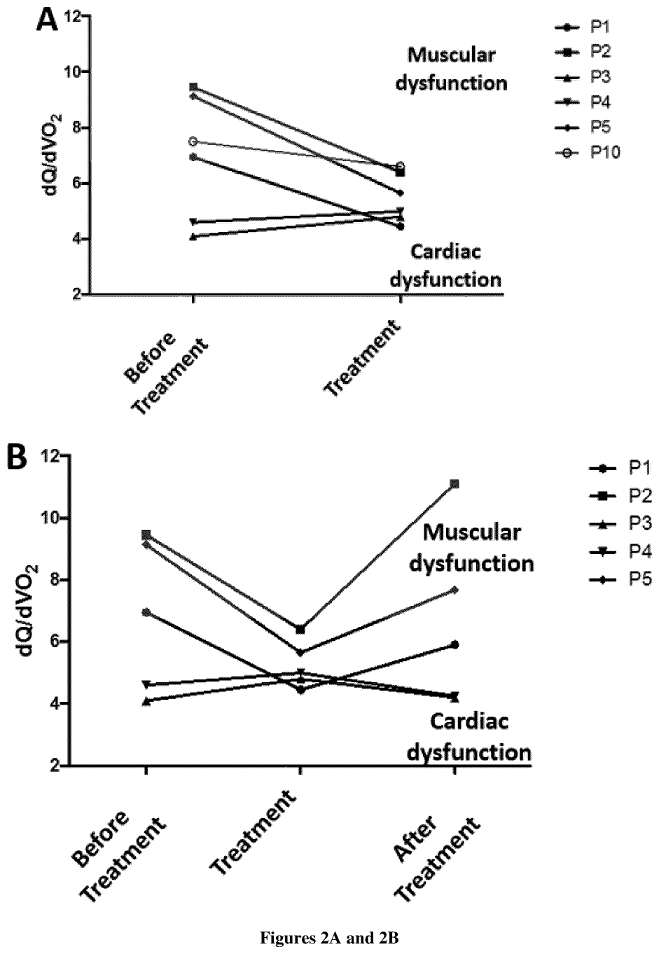

A sub-maximal exercise test was performed in 5 out of 6 subjects older than 6 years old who were able to perform age-appropriate muscle assessments at Baseline and during the follow-up. Five patients had an abnormal exercise test, with a high dQ/dVO2 ratio due to impaired oxygen uptake by peripheral muscle in patients P1, P2, P5 and P10 (mean at 7, 9.5, 9.1 and 7.5 respectively), and with a low dQ/dVO2 ratio due to cardiac failure in patients P3 (mean at 4) (FIG. 2A). P10 developed an acute RM (max. CK levels 75 000 U/L) following the Baseline test, and the test was not subsequently proposed under treatment. Among the 5 patients who realized effort tests at Baseline and during the follow-up, any experimented RM. The four patients with abnormal peripheral muscle improved their muscle performance with a decrease of dQ/dVO2 ratio after initiation of treatment (global mean at 5.8). At contrary, the patient with cardiac failure increased his ratio under treatment (mean at 4.8), confirming an improvement of his cardiac function (FIG. 2B). Among patients who had stopped HCQ treatment, all patients suffered from an alteration ratio. P4, with normal ratio prior treatment, presented an abnormal ratio after discontinuation of HCQ treatment.

Any allergy symptoms have been observed with HCQ treatment. Side effects associated with HCQ were abdominal pain in patients 5 and 6.

Electroretinogramms and eye fundus were normal at 12 months of treatment for all the patients tested.

Inflammatory Cytokines Levels in Plasma

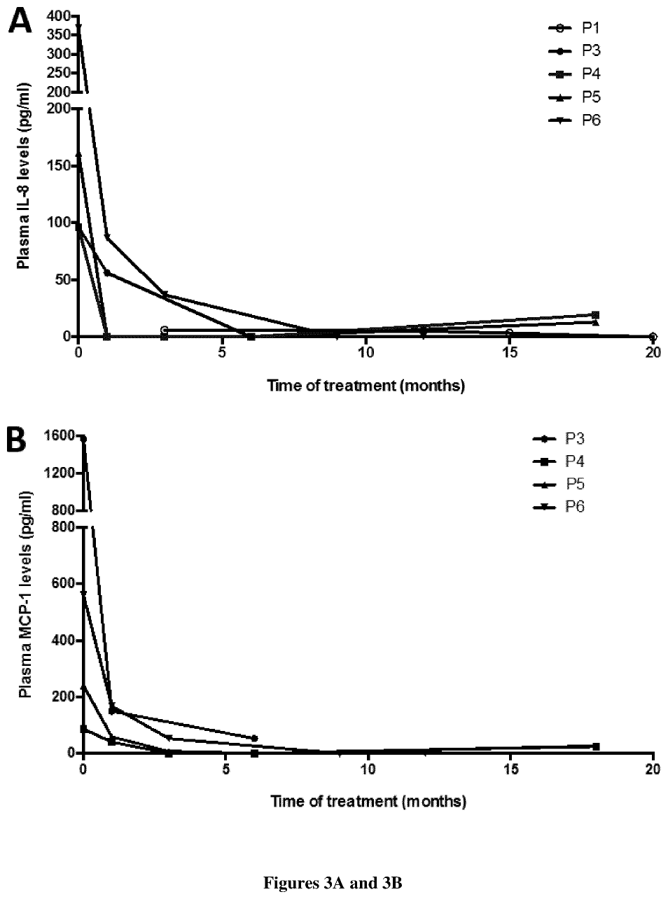

In all patients tested, HCQ reduced the plasma levels of inflammatory cytokines IL8 and MCP-1 (FIGS. 3A-3B) suggesting a reduction in their inflammatory status. These results suggest a beneficial action of HCQ on patients' inflammation.

Define the Mechanisms of Action of HCQ According to the Concentrations of the Molecule in Patient Myoblasts

Because we hypothesized the existence of a dose-effect relationship requiring the definition of a therapeutic window in patients, we sought to define the mechanisms of action of HCQ according to the concentrations of the molecule in patient myoblasts.

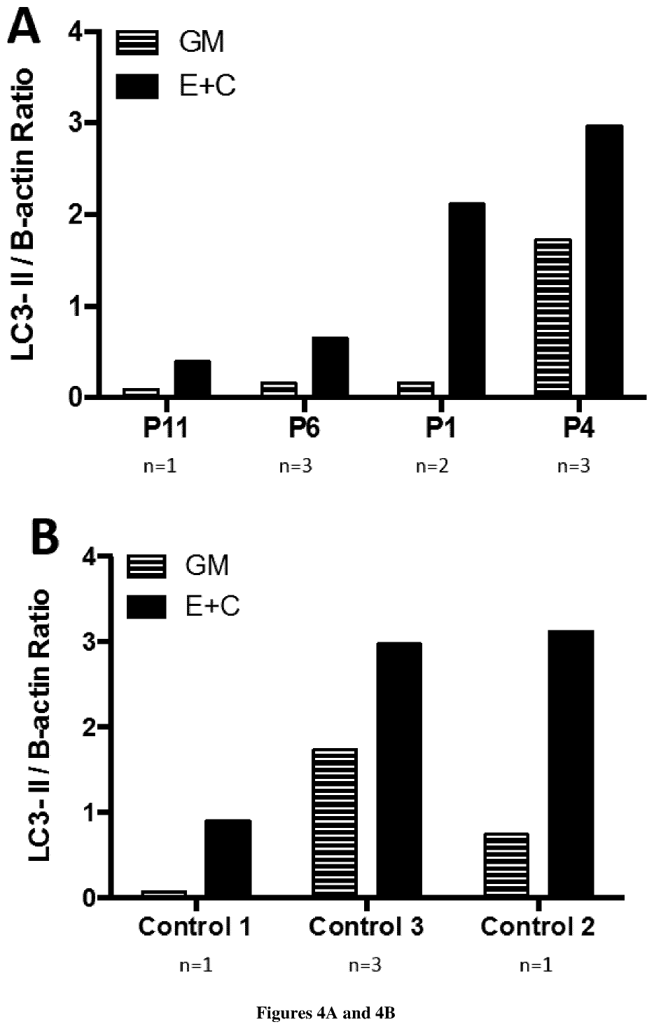

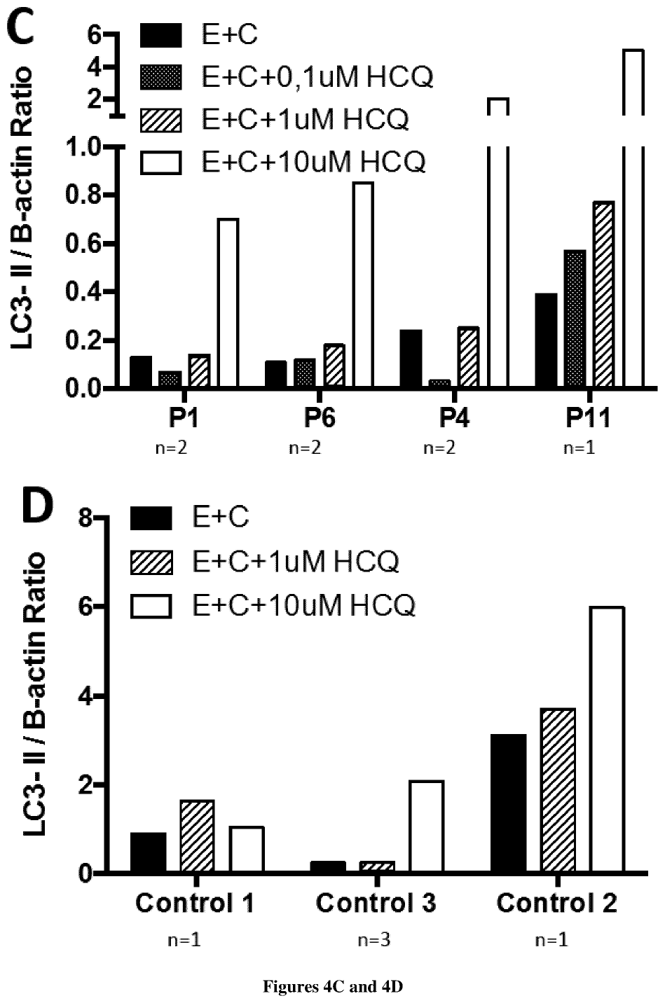

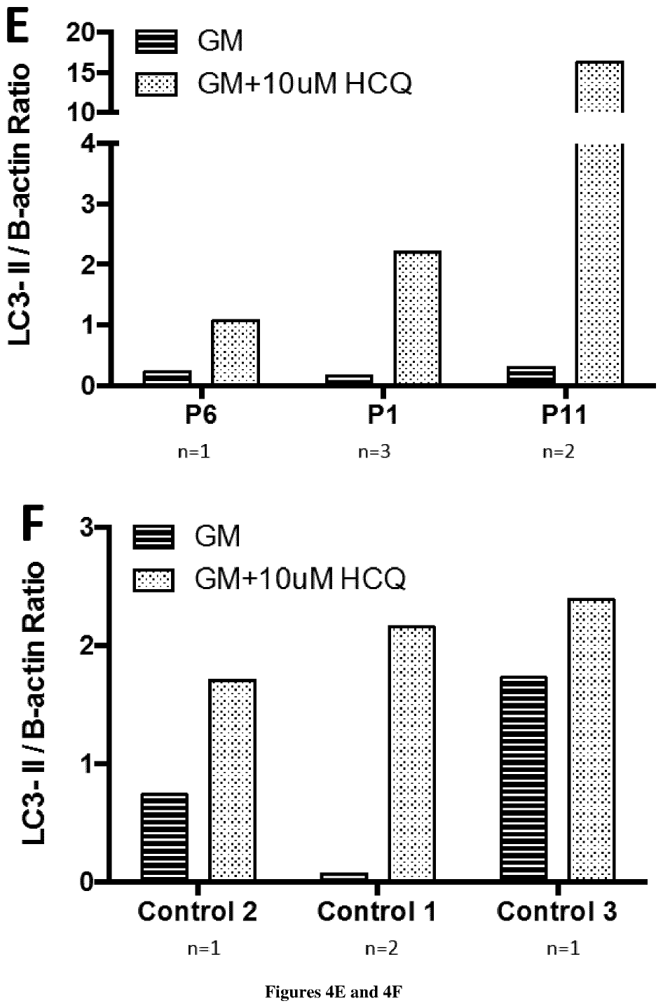

In vitro studies were performed on myoblasts from 4 patients (P1, P4, P6 in Table 1, and another patient untreated P11) and from healthy controls after RM induction by myoblasts starvation (EBSS medium) in presence of TLR9 agonist (CpGA). We previously showed that in patient myoblasts, induction of RM is associated with an autophagy blockage. As blockage of autophagy is a well-known mechanism of action of HCQ in various models, we studied the impact of HCQ treatment on autophagy by measuring LCII/B-actin ratio (immunoblot). Our results show that HCQ potentiates the autophagy blockage induced by the immunometabolic stress both in control and patients myoblasts as LC3-II accumulated (FIGS. 4A-4F). This effect is dose dependant as HCQ 10 μM concentration potentiates LC3-II accumulation induced by immunometabolic stress condition whereas lower concentrations (0.1 and 1 μM) of HCQ are less deleterious.

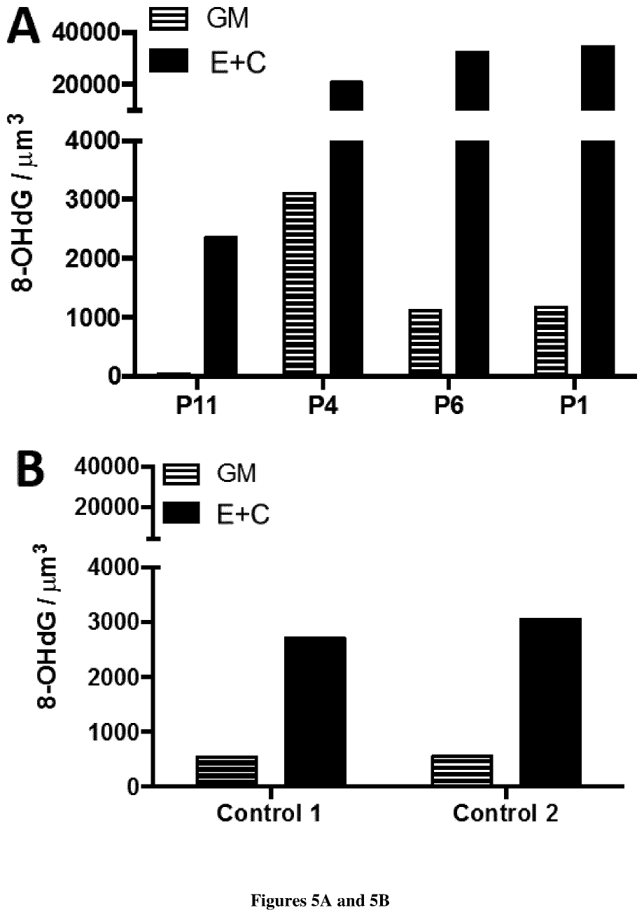

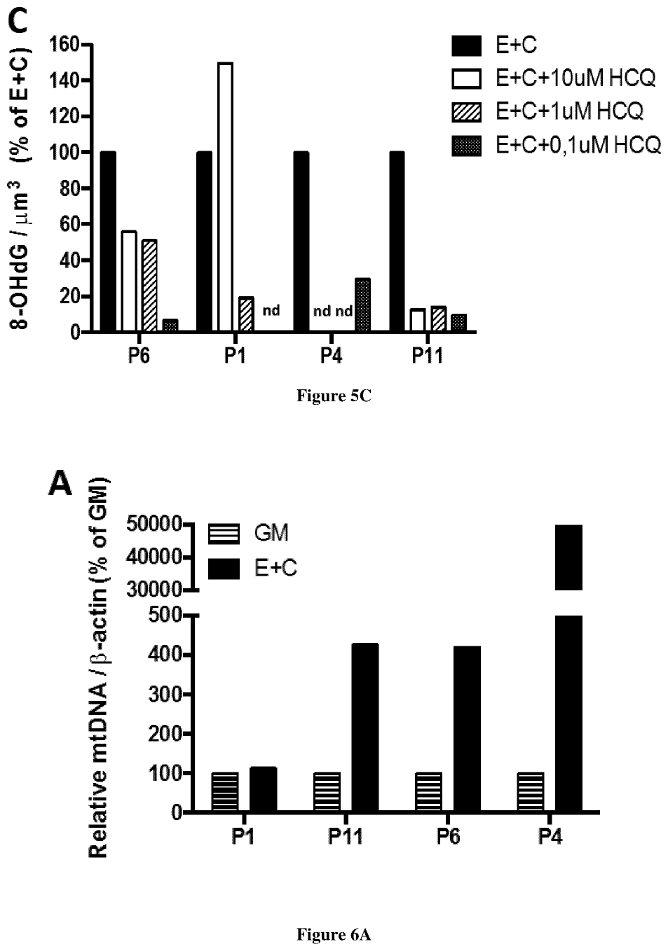

We showed that in Lipin-1 deficient myoblasts, immunometabolic stress condition was associated with accumulation of oxDNA (FIGS. 5A-5C). In four patients (P1, P4, P6 and P11), HCQ treatment prevents this accumulation (FIG. 5C).

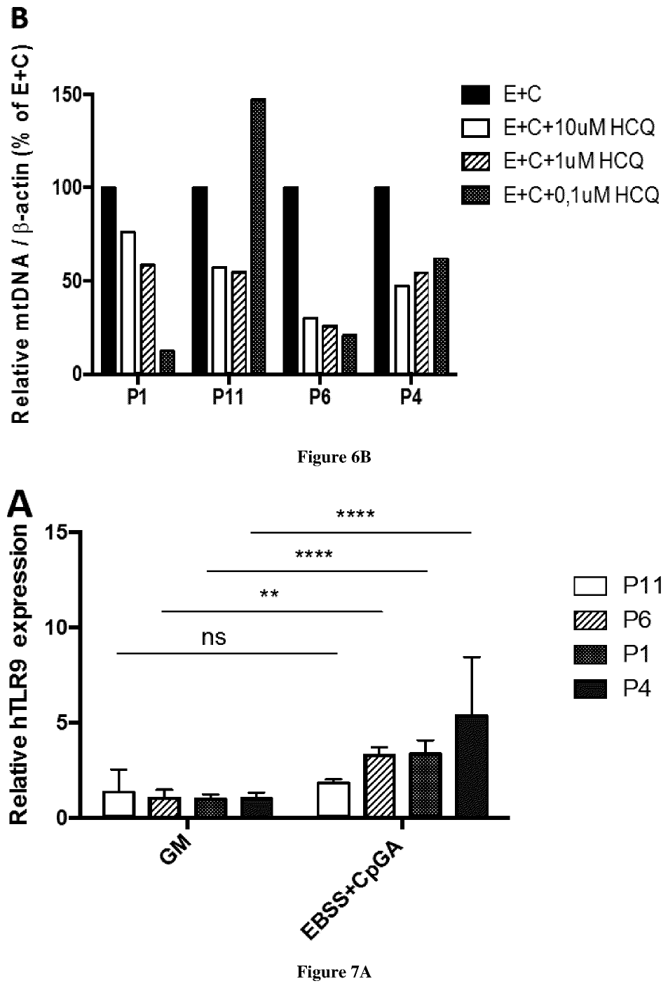

In the same way, we showed that in Lipin-1 deficient myoblasts, immunometabolic stress condition was associated with accumulation of mtDNA (FIG. 6A). In four patients (P1, P4, P6 and P11), HCQ treatment prevents this accumulation (FIG. 6B). However these results need to be reproduced as in other manipulations we did not obtain an accumulation of mtDNA in immunomatabolic stress conditions (data not shown).

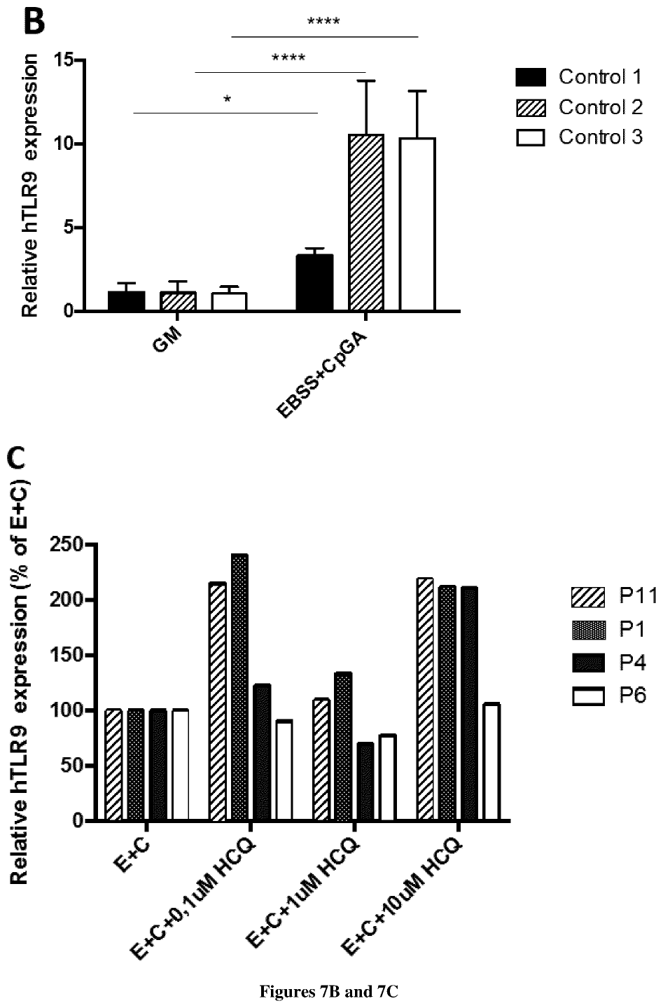

Because of mtDNA was the natural ligand of TLR9, we were interested in this TLR signaling pathway. First, we measured TLR9 expression in myoblasts of patients and controls. Immunometabolic stress condition increased TLR9 expression compared to basal condition in both primary patient and control myoblasts (FIGS. 7A-7B). HCQ treatment had an variable effect on TLR9 expression under stress condition (FIG. 7C) but this molecule could not decrease TLR9 expression as described in literature. Functional studies of TLR9 would be established in order to complete these primary results.

Taking together our results strongly suggest a dose dependent effect of HCQ with deleterious effect on autophagy at 10 μM, and a beneficial effect on oxDNA without negative impact on autophagy at 1 μM or 0.1 μM depending on the patient.

Finally, as we had already described a variability in the occurrence of RM under HCQ treatment in patients, an other hypothesis was the existence of an patient-dependent effect. In order to evaluate this hypothesis, we also established a pharmacogenetic analysis of HCQ treatment by sequencing genes encoding drug metabolism enzymes (CYP450): CYP3A4/CYP2C8 (responsible for 80% of the HCQ metabolism), and CYP3A5/CYP2D6 (Data not shown). Our first results showed that there is no difference in pharmacogenetic analysis between patients. Then, we were interested in the dosage og HCQ in vitro in order to analyse the HCQ incorporation in primary patients myoblasts. We observed a decrease of HCQ quantity in culture supernatant, and a detection of HCQ in myoblasts after cellular lysis (data not shown). Our results confirm that HCQ incorporation is effective in primary myoblasts. Analysis of HCQ dosage in different patient myoblasts would be necessary in order to perform the hypothesis of a patient-dependent effect of HCQ.

Discussion

Based on our first results with TLR9 antagonists and in absence of animal models, we proposed a compassionate trial to evaluate the safety and the impact of HCQ treatment in Lipin-1 disease, routinely used in children and adults in other indications, and to define the mechanism of action of HCQ on myoblasts. HCQ was preferred over chloroquine because of less cardiac side effects⁵⁹.

HCQ significantly reduced levels of inflammatory cytokines in plasma of the patients. When treated, seven on ten patients did not experience any new episode of RM. Upon discontinuation of treatment for one patient, a reminiscence of severe RM was observed, reversible upon resuming HCQ treatment. Muscle outcome measures improved under HCQ treatment: muscle pain, quality of life, walking capacity in 6 MWT distance, step climbing in 3 MST steps, and exercise test. The patient who exhibited slight left ventricular dysfunction at rest improved his LVEF from 45% to 63%. Finally, HCQ was well tolerated in patients. Based on our data resulting in the first three patients, we obtained the Orphan Drug Designation from the European Medicines Agency for Hydroxychloroquine (EMA/OD/177/17) in January 2018.

However, two next patients presented one and two RM respectively, in a context of gastroenteritis. Because they had the highest concentrations of HCQ in plasma, we suspected an overdose of the drug, and proposed to strictly monitor the plasma levels of HCQ in order to obtain a therapeutic window between 100 and 700 ng/ml, using a soluble form for the youngest patients. This proposition goes against that proposed in systemic lupus erythematosus (SLE) in which blood concentrations of HCQ above 750 ng/mL are associated with significant improvement in refractory cutaneous LE⁶⁰. Likewise, autophagy has emerged as a key homeostatic mechanism in SLE^{61,62}. Compared with healthy controls, Tolerogenic T lymphocytes of patients with SLE exhibit activated autophagy, resulting in deregulation of adaptive immunity. CQ autophagy inhibition in vitro rebalance the immune response in SLE patients and mice, and ameliorated SLE⁶². Paradoxically, long-term side effects of HCQ including cardiomyopathy^{63,64} and vacuolar myopathy⁵⁹, that are potentially reversible after stopping the drug⁵⁹, may be due to the iatrogenic blockage of autophagy. Indeed, a microscopic study of muscle showed typical vacuolar myopathy with myeloid bodies and/or curvilinear bodies, indicative of impaired autophagy⁶⁵, while microscopic features of chloroquine cardiotoxicity are similar to those of Fabry disease with sarcoplasmic myelinoid bodies⁶⁶.

If we used HCQ for its anti-TLR9 action in Lipin-1 disease, it could be surprising to propose such drug that is known to also inhibit autophagy. The dose-effect of the drug observed in our patients underlines the prudence of use of the drug and the need to define the mechanisms of actions of HCQ in myoblasts as still now they remain unclear and not or poorly studied in muscle (see below). We confirmed in myoblasts the existence of a dose-effect relationship for the different actions of HCQ. Low to high concentrations (0.1 to 10 μM respectively) of HCQ normalized oxDNA levels in myoblasts but high concentration (10 μM) of this molecule worsened the blockage of autophagy already described in patients' myoblasts under stress condition, evoking the clinical overdosage in patients.

If the mechanisms of actions of HCQ/CQ remain unclear, several contradictory effects are described: i) the inhibition of endolysosomal acidification⁶⁷⁻⁶⁹, acidic pH being a prerequisite for autophagy and endosomal TLR activation⁶⁷⁻⁶⁹, ii) thus a blockage of autophagy at the final step of phagolysosomes. However several studies are contradictory as, in contrast to bafilomycin, and despite CQ accumulation in acidic compartments in cells⁷⁰, CQ/HCQ at low or high concentration does not affect endosomal and lysosomal acidification in different studies⁷¹⁻⁷⁴. Mauthe et al recently showed that CQ inhibits autophagic flux by directly impairing autophagosome fusion with lysosome independently of the pH⁷⁴, and induces an autophagy-independant severe disorganisation of the Golgi and endo-lysosomal systems, contributing to the fusion impairment. Other actions of HCQ are iii) anti-TLR9 activity, by the inhibition of endolysosomal acidification, but also by an interaction with double-stranded DNA that links to TLR9, affecting its conformation and availability for TLR binding sites^{72 {Cohen, 1965 #4929,73,75}. Finally, CQ was also described to i) decrease TLR9 expression, ii) activate DNAse II⁷⁶, a main acidic endonuclease for destruction of DNA that is activated in lysosomes, and AMPK phosphorylation^{77,78}. All these actions lead to a reduction of mtDNA and ROS and an increase of ATP in different models, such as stressed rats⁷⁶, mice and human renal tubular cells in a diabetic environment⁷⁸, or myotube cells⁷⁷ in with a concomitant decrease of intracellular calcium concentration was observed. The increase of AMPK phosphorylation by CQ also improves mitochondrial biogenesis via PGC-1a, mitochondrial mass (Tom20), balanced fusion/fission protein (Mfn1/Drp1) expression, with a consecutive decrease of ROS and 8-OHdG content, mitochondrial fragmentation and cell apoptosis⁷⁸. Inversely, it has been also shown that CQ inhibits mitochondrial proteins such as cytochrome c oxidase, with decrease in ATP production⁷⁹, and that it is an uncoupling agent that induces metabolic stress⁸⁰.

Interestingly, the decrease in DAG by Lipin dysfunction, required for the biosynthesis of phospholipids⁸¹, may affect the cell cycle in S phase, as phospholipids are accumulated in the S phase⁸². Earlier studies reported a possible involvement of phospholipids in DNA replication (inhibition) in mitochondria and in nucleus through interaction with DNA polymerase (i.e., cardiolipin, PA, phosphatidylglycerol, and phosphatidylinositol)^{83,84}. Notably, Lipin can regulate the syntheses of phosphatidylcholine and other phospholipids by repressing key genes of the biosynthesis pathway in yeast⁸⁵. Thus, it is possible that the abnormal PA/DAG in Lipin-kd models inhibit the interaction between DNA polymerases and phospholipids, thereby causing DNA replication stress by generating incompletely replicated DNA.

Gastrointestinal or respiratory virus frequently described in Lipin-1-related RM underline the role of virus mediated by TLR9^{86,87}. Notably, it has been shown in pah1Δ yeast that proliferation and expansion of the ER with augmentation of PL content^{88,89} facilitate RNA virus replication^{90,91} which take advantage of expanded membranes surface and lipids^{90,92,93}. Thus, apart HCQ to prevent RM flares, we recommend to administrate anti-inflammatory treatments such as steroids since the first signs of acute RM in order to control the inflammatory cascade, especially since myotoxic cytokines damage skeletal muscle⁹⁴. As a prevention, we also recommend to vaccinate Lipin-1 related patients with all available vaccines, the risk of decompensation being very high during viral epidemics^{95,96} and rare after vaccines⁹⁷.

In conclusion, we describe the first human experience with HCQ for Lipin-1 disease, a fatal disease in infancy. In a relevant pre-clinical cell model, HCQ normalized oxDNA levels, which could in turn reduce TLR9 activation and inflammation, but with deleterious effect at high concentrations of HCQ. Our results allow us to propose HCQ as a long-term treatment to prevent further relapses in this severe disease, with a close concentration monitoring of the drug to avoid excessive autophagy blockage observed at high HCQ concentrations, and subsequent risk of RM. These recommendations are the opposite of those proposed in lupus.

Tables:

TABLE 1
Clinical description of Lipin1 patients

| | | | Number of | | | |
|---|---|---|---|---|---|---|
| | | | Myolysis | | | |
| | | Age at | (CK > 6 000 | Chronic | | Aminoacid |
| Patients | Sex | treatment | U/L) | symptoms | Mutations | change |
| P1 (EA) | M | 8 y | 6 | Muscle pain | c.1162C > T | p.Arg388X |
| | | | | Fatigability | c.1162C > T | p.Arg388X |
| P2 (EA) | M | 13 y | 5 | Muscle pain | c.1162C > T | p.Arg388X |
| | | | | Fatigability | c.1162C > T | p.Arg388X |
| P3 (FN) | M | 17 y | 1 | Muscle pain | c.1441 + 2T > C | p.Asn417LysfsX22 |
| | | | | Fatigability | c.2295- | p.Glu766_Ser838del |
| | | | | Cardiac failure | 866_2410-30del | |
| | | | | 17 y | | |
| P4 (FE) | M | 12 y | 3 | Muscle pain | c.1441 + 2T > C | p.Asn417LysfsX22 |
| | | | | Fatigability | c.2295- | p.Glu766_Ser838del |
| | | | | | 866_2410-30del | |
| P5 (FJ) | F | 8 y | 3 | Muscle pain | c.1441 + 2T > C | p.Asn417LysfsX22 |
| | | | | Fatigability | c.2295- | p.Glu766_Ser838del |
| | | | | | 866_2410-30del | |
| P6 (FA) | F | 4 y | 2 | No | c.1642del | p.Ser548Profs * 45 |
| | | | | | c.1642del | p.Ser548Profs * 45 |
| P7 (FA) | F | 2 y | 0 | No | c.1642del | p.Ser548Profs * 45 |
| | | | | | c.1642del | p.Ser548Profs * 45 |
| P8 (DK) | F | 9 y | 2 | Syndromic | C.181C > T | p.Arg61X |
| | | | | mental | C.181C > T | p.Arg61X |
| | | | | retardation | | |
| P9 (RK) | M | 5 y | 4 | No | c.2295- | p.Glu766_Ser838del |
| | | | | | 863_2410-27del | p.Glu766_Ser838del |
| | | | | | c.2295- | |
| | | | | | 863_2410-27del | |
| P10 (FA) | M | 18 y | 3 | Muscle pain | c.377_380dup | p.Met128GlnfsX45 |
| | | | | Fatigability | c.2295- | p.Glu766_Ser838del |
| | | | | | 863_2410-27del | |

TABLE 2
Number of rhabdomyolysis under treatment and
concentration of Hydroxychloroquine in plasma

| | Age at the | | Number of | Number of | | |
|---|---|---|---|---|---|---|
| | beginning of | Treatment | severe RM | severe RM | Plasma HCQ | |
| | treatment | duration | (CPK > 10k U/L) | (CPK > 10k U/L) | (μg/L) mean | |
| Patient | (years) | (months) | before treatment | during treatment | (median) | |
| P1 | 7 | 39 | 6 | 0 | 318 | 325) |
| P2 | 13 | 37 | 5 | 0 | 107 | (107) |
| P3 | 17 | 12 | 1 | 0 | 724 | (854) |
| P4 | 12 | 12 | 3 | 0 | 627 | (597) |
| P5 | 8 | 12 | 3 | 1 | 1048 | (955) |
| P6 | 4 | 41 | 2 | 3 | 1040 | (1092) |
| P7 | 2 | 25 | 0 | 4 | 290 | (205) |
| P8 | 9 | 41 | 2 | 0 | 610 | (610) |
| P9 | 4 | 14 | 4 | 0 | 407 | (367) |
| P10 | 20 | 16 | 3 | 0 | 752 | (733) |

### REFERENCES

Throughout this application, various references describe the state of the art to which this invention pertains. The disclosures of these references are hereby incorporated by reference into the present disclosure.
(1) Zeharia, A.; Shaag, A.; Houtkooper, R. H.; Hindi, T.; de Lonlay, P.; Erez, G.; Hubert, L.; Saada, A.; de Keyzer, Y.; Eshel, G.; Vaz, F. M.; Pines, O.; Elpeleg, O. Mutations in LPIN1 cause recurrent acute myoglobinuria in childhood. Am J Hum Genet 2008, 83, 489-494.
(2) Michot, C.; Hubert, L.; Brivet, M.; De Meirleir, L.; Valayannopoulos, V.; Muller-Felber, W.; Venkateswaran, R.; Ogier, H.; Desguerre, I.; Altuzarra, C.; Thompson, E.; Smitka, M.; Huebner, A.; Husson, M.; Horvath, R.; Chinnery, P.; Vaz, F. M.; Munnich, A.; Elpeleg, O.; Delahodde, A.; de Keyzer, Y.; de Lonlay, P. LPIN1 gene mutations: a major cause of severe rhabdomyolysis in early childhood. Hum Mutat 2010, 31, E1564-1573.
(3) Michot, C.; Hubert, L.; Romero, N. B.; Gouda, A.; Mamoune, A.; Mathew, S.; Kirk, E.; Viollet, L.; Rahman, S.; Bekri, S.; Peters, H.; McGill, J.; Glamuzina, E.; Farrar, M.; von der Hagen, M.; Alexander, I. E.; Kirmse, B.; Barth, M.; Laforet, P.; Benlian, P.; Munnich, A.; Jeanpierre, M.; Elpeleg, O.; Pines, O.; Delahodde, A.; de Keyzer, Y.; de Lonlay, P. Study of LPIN1, LPIN2 and LPIN3 in rhabdomyolysis and exercise-induced myalgia. J Inherit Metab Dis 2012.
(4) Bergounioux, J.; Brassier, A.; Rambaud, C.; Bustarret, O.; Michot, C.; Hubert, L.; Arnoux, J. B.; Laquerriere, A.; Bekri, S.; Galene-Gromez, S.; Bonnet, D.; Hubert, P.; de Lonlay, P. Fatal rhabdomyolysis in 2 children with LPIN1 mutations. J Pediatr 2012, 160, 1052-1054.
(5) Schweitzer, G. G.; Collier, S. L.; Chen, Z.; Eaton, J. M.; Connolly, A. M.; Bucelli, R. C.; Pestronk, A.; Harris, T. E.; Finck, B. N. Rhabdomyolysis-Associated Mutations in Human LPIN1 Lead to Loss of Phosphatidic Acid Phosphohydrolase Activity. JIMD Rep 2015.
(6) Stepien, K. M.; Schmidt, W. M.; Bittner, R. E.; O'Toole, O.; McNamara, B.; Treacy, E. P. Long-term outcomes in a 25-year-old female affected with lipin-1 deficiency. JIMD Rep 2019, 46, 4-10.
(7) Pichler, K.; Scholl-Buergi, S.; Birnbacher, R.; Freilinger, M.; Straub, S.; Brunner, J.; Zschocke, J.; Bittner, R. E.; Karall, D. A novel therapeutic approach for LPIN1 mutation-associated rhabdomyolysis—The Austrian experience. Muscle Nerve 2015, 52, 437-439.
(8) Nunes, D.; Nogueira, C.; Lopes, A.; Chaves, P.; Rodrigues, E.; Cardoso, T.; Leao Teles, E.; Vilarinho, L. LPIN1 deficiency: A novel mutation associated with different phenotypes in the same family. Mol Genet Metab Rep 2016, 9, 29-30.
(9) Meijer, I. A.; Sasarman, F.; Maftei, C.; Rossignol, E.; Vanasse, M.; Major, P.; Mitchell, G. A.; Brunel-Guitton, C. LPIN1 deficiency with severe recurrent rhabdomyolysis and persistent elevation of creatine kinase levels due to chromosome 2 maternal isodisomy. Mol Genet Metab Rep 2015, 5, 85-88.
(10) Kok, B. P.; Kienesberger, P. C.; Dyck, J. R.; Brindley, D. N. Relationship of glucose and oleate metabolism to cardiac function in lipin-1 deficient (fld) mice. J Lipid Res 2012, 53, 105-118.
(11) Legendre, A.; Khraiche, D.; Ou, P.; Mauvais, F. X.; Madrange, M.; Guemann, A. S.; Jais, J. P.; Bonnet, D.; Hamel, Y.; de Lonlay, P. Cardiac function and exercise adaptation in 8 children with LPIN1 mutations. Mol Genet Metab 2018, 123, 375-381.
(12) Donkor, J.; Sariahmetoglu, M.; Dewald, J.; Brindley, D. N.; Reue, K. Three mammalian lipins act as phosphatidate phosphatases with distinct tissue expression patterns. J Biol Chem 2007, 282, 3450-3457.
(13) Harris, T. E.; Finck, B. N. Dual function lipin proteins and glycerolipid metabolism. Trends Endocrinol Metab 2011, 22, 226-233.
(14) Reue, K. The role of lipin 1 in adipogenesis and lipid metabolism. Novartis Found Symp 2007, 286, 58-68; discussion 68-71, 162-163, 196-203.
(15) Phan, J.; Peterfy, M.; Reue, K. Biphasic expression of lipin suggests dual roles in adipocyte development. Drug News Perspect 2005, 18, 5-11.
(16) Mitra, M. S.; Chen, Z.; Ren, H.; Harris, T. E.; Chambers, K. T.; Hall, A. M.; Nadra, K.; Klein, S.; Chrast, R.; Su, X.; Morris, A. J.; Finck, B. N. Mice with an adipocyte-specific lipin 1 separation-of-function allele reveal unexpected roles for phosphatidic acid in metabolic regulation. Proc Natl Acad Sci USA 2012.
(17) Finck, B. N.; Gropler, M. C.; Chen, Z.; Leone, T. C.; Croce, M. A.; Harris, T. E.; Lawrence, J. C., Jr.; Kelly, D. P. Lipin 1 is an inducible amplifier of the hepatic PGC-1alpha/PPARalpha regulatory pathway. Cell Metab 2006, 4, 199-210.
(18) Pelosi, M.; Testet, E.; Le Lay, S.; Dugail, I.; Tang, X.; Mabilleau, G.; Hamel, Y.; Madrange, M.; Blanc, T.; Odent, T.; McMullen, T. P. W.; Alfo, M.; Brindley, D. N.; de Lonlay, P. Normal human adipose tissue functions and differentiation in patients with biallelic LPIN1 inactivating mutations. J Lipid Res 2017.
(19) Michot, C.; Mamoune, A.; Vamecq, J.; Viou, M. T.; Hsieh, L. S.; Testet, E.; Laine, J.; Hubert, L.; Dessein, A. F.; Fontaine, M.; Ottolenghi, C.; Fouillen, L.; Nadra, K.; Blanc, E.; Bastin, J.; Candon, S.; Pende, M.; Munnich, A.; Smahi, A.; Djouadi, F.; Carman, G. M.; Romero, N.; de Keyzer, Y.; de Lonlay, P. Combination of lipid metabolism alterations and their sensitivity to inflammatory cytokines in human lipin-1-deficient myoblasts. Biochim Biophys Acta 2013, 1832, 2103-2114.
(20) Janeway, C. A., Jr.; Medzhitov, R. Innate immune recognition. Annu Rev Immunol 2002, 20, 197-216.
(21) Zhang, P.; Verity, M. A.; Reue, K. Lipin-1 Regulates Autophagy Clearance and Intersects with Statin Drug Effects in Skeletal Muscle. Cell Metab 2014.
(22) Alshudukhi, A. A.; Zhu, J.; Huang, D.; Jama, A.; Smith, J. D.; Wang, Q. J.; Esser, K. A.; Ren, H. Lipin-1 regulates Bnip3-mediated mitophagy in glycolytic muscle. FASEB J 2018, 32, 6796-6807.
(23) Sasser, T.; Qiu, Q. S.; Karunakaran, S.; Padolina, M.; Reyes, A.; Flood, B.; Smith, S.; Gonzales, C.; Fratti, R. A. Yeast lipin 1 orthologue pah1p regulates vacuole homeostasis and membrane fusion. J Biol Chem 2012, 287, 2221-2236.
(24) Palamiuc, L.; Ravi, A.; Emerling, B. M. Phosphoinositides in autophagy: current roles and future insights. FEBS J 2019.
(25) Jahn, R.; Sudhof, T. C. Membrane fusion and exocytosis. Annu Rev Biochem 1999, 68, 863-911.
(26) Jaber, N.; Mohd-Naim, N.; Wang, Z.; DeLeon, J. L.; Kim, S.; Zhong, H.; Sheshadri, N.; Dou, Z.; Edinger, A. L.; Du, G.; Braga, V. M.; Zong, W. X. Vps34 regulates Rab7 and late endocytic trafficking through recruitment of the GTPase-activating protein Armus. J Cell Sci 2016, 129, 4424-4435.
(27) Wang, K.; Klionsky, D. J. Mitochondria removal by autophagy. Autophagy 2011, 7, 297-300.
(28) Masiero, E.; Agatea, L.; Mammucari, C.; Blaauw, B.; Loro, E.; Komatsu, M.; Metzger, D.; Reggiani, C.; Schiaffino, S.; Sandri, M. Autophagy is required to maintain muscle mass. Cell Metab 2009, 10, 507-515.
(29) Masiero, E.; Sandri, M. Autophagy inhibition induces atrophy and myopathy in adult skeletal muscles. Autophagy 2010, 6, 307-309.
(30) Hamanaka, R. B.; Chandel, N. S. Mitochondrial reactive oxygen species regulate cellular signaling and dictate biological outcomes. Trends Biochem Sci 2010, 35, 505-513.
(31) Ravi, S.; Pena, K. A.; Chu, C. T.; Kiselyov, K. Biphasic regulation of lysosomal exocytosis by oxidative stress. Cell Calcium 2016, 60, 356-362.
(32) Zhang, X.; Cheng, X.; Yu, L.; Yang, J.; Calvo, R.; Patnaik, S.; Hu, X.; Gao, Q.; Yang, M.; Lawas, M.; Delling, M.; Marugan, J.; Ferrer, M.; Xu, H. MCOLN1 is a ROS sensor in lysosomes that regulates autophagy. Nat Commun 2016, 7, 12109.
(33) Twig, G.; Hyde, B.; Shirihai, O. S. Mitochondrial fusion, fission and autophagy as a quality control axis: the bioenergetic view. Biochim Biophys Acta 2008, 1777, 1092-1097.
(34) Kubli, D. A.; Gustafsson, A. B. Mitochondria and mitophagy: the yin and yang of cell death control. Circ Res 2012, 111, 1208-1221.
(35) Green, D. R.; Kroemer, G. The pathophysiology of mitochondrial cell death. Science 2004, 305, 626-629.
(36) Fang, C.; Wei, X.; Wei, Y. Mitochondrial DNA in the regulation of innate immune responses. Protein Cell 2016, 7, 11-16.
(37) Zhang, Q.; Raoof, M.; Chen, Y.; Sumi, Y.; Sursal, T.; Junger, W.; Brohi, K.; Itagaki, K.; Hauser, C. J. Circulating mitochondrial DAMPs cause inflammatory responses to injury. Nature 2010, 464, 104-107.
(38) Zhang, J. Z.; Liu, Z.; Liu, J.; Ren, J. X.; Sun, T. S. Mitochondrial DNA induces inflammation and increases TLR9/NF-kappaB expression in lung tissue. Int J Mol Med 2014, 33, 817-824.
(39) Oka, T.; Hikoso, S.; Yamaguchi, O.; Taneike, M.; Takeda, T.; Tamai, T.; Oyabu, J.; Murakawa, T.; Nakayama, H.; Nishida, K.; Akira, S.; Yamamoto, A.; Komuro, I.; Otsu, K. Mitochondrial DNA that escapes from autophagy causes inflammation and heart failure. Nature 2012, 485, 251-255.
(40) Bao, W.; Xia, H.; Liang, Y.; Ye, Y.; Lu, Y.; Xu, X.; Duan, A.; He, J.; Chen, Z.; Wu, Y.; Wang, X.; Zheng, C.; Liu, Z.; Shi, S. Toll-like Receptor 9 Can be Activated by Endogenous Mitochondrial DNA to Induce Podocyte Apoptosis. Sci Rep 2016, 6, 22579.
(41) Knochel, J. P.; Moore, G. E. Rhabdomyolysis in Malaria. N Engl J Med 1993, 329, 1206-1207.
(42) Pedersen, B. K.; Steensberg, A.; Fischer, C.; Keller, C.; Ostrowski, K.; Schjerling, P. Exercise and cytokines with particular focus on muscle-derived IL-6. Exerc Immunol Rev 2001, 7, 18-31.
(43) Pereira, B. C.; Pauli, J. R.; Antunes, L. M.; de Freitas, E. C.; de Almeida, M. R.; de Paula Venancio, V.; Ropelle, E. R.; de Souza, C. T.; Cintra, D. E.; Papoti, M.; da Silva, A. S. Overtraining is associated with DNA damage in blood and skeletal muscle cells of Swiss mice. BMC Physiol 2013, 13, 11.
(44) Fox, R. Anti-malarial drugs: possible mechanisms of action in autoimmune disease and prospects for drug development. Lupus 1996, 5 Suppl 1, S4-10.
(45) Wozniacka, A.; Lesiak, A.; Boncela, J.; Smolarczyk, K.; McCauliffe, D. P.; Sysa-Jedrzejowska, A. The influence of antimalarial treatment on IL-1beta, IL-6 and TNF-alpha mRNA expression on UVB-irradiated skin in systemic lupus erythematosus. Br J Dermatol 2008, 159, 1124-1130.
(46) Sun, S.; Rao, N. L.; Venable, J.; Thurmond, R.; Karlsson, L. TLR7/9 antagonists as therapeutics for immune-mediated inflammatory disorders. Inflamm Allergy Drug Targets 2007, 6, 223-235.
(47) Pons-Estel, G. J.; Alarcon, G. S.; McGwin, G., Jr.; Danila, M. I.; Zhang, J.; Bastian, H. M.; Reveille, J. D.; Vila, L. M.; Lumina Study, G. Protective effect of hydroxychloroquine on renal damage in patients with lupus nephritis: LXV, data from a multiethnic US cohort. Arthritis Rheum 2009, 61, 830-839.
(48) Goemans, N.; Klingels, K.; van den Hauwe, M.; Boons, S.; Verstraete, L.; Peeters, C.; Feys, H.; Buyse, G. Six-minute walk test: reference values and prediction equation in healthy boys aged 5 to 12 years. PLoS One 2013, 8, e84120.
(49) Kierkegaard, M.; Tollback, A. Reliability and feasibility of the six minute walk test in subjects with myotonic dystrophy. Neuromuscul Disord 2007, 17, 943-949.
(50) Wegrzynowska-Teodorczyk, K.; Mozdzanowska, D.; Josiak, K.; Siennicka, A.; Nowakowska, K.; Banasiak, W.; Jankowska, E. A.; Ponikowski, P.; Wozniewski, M. Could the two-minute step test be an alternative to the six-minute walk test for patients with systolic heart failure? Eur J Prev Cardiol 2016, 23, 1307-1313.
(51) Lang, R. M.; Bierig, M.; Devereux, R. B.; Flachskampf, F. A.; Foster, E.; Pellikka, P. A.; Picard, M. H.; Roman, M. J.; Seward, J.; Shanewise, J.; Solomon, S.; Spencer, K. T.; St John Sutton, M.; Stewart, W.; American Society of Echocardiography's, N.; Standards, C.; Task Force on Chamber, Q.; American College of Cardiology Echocardiography, C.; American Heart, A.; European Association of Echocardiography, E. S. o. C. Recommendations for chamber quantification. Eur J Echocardiogr 2006, 7, 79-108.
(52) Haller, R. G. Oxygen utilization and delivery in metabolic myopathies. Ann Neurol 1994, 36, 811-813.
(53) Miller, D. R.; Fiechtner, J. J.; Carpenter, J. R.; Brown, R. R.; Stroshane, R. M.; Stecher, V. J. Plasma hydroxychloroquine concentrations and efficacy in rheumatoid arthritis. Arthritis Rheum 1987, 30, 567-571.
(54) Leon, J.; Sakumi, K.; Castillo, E.; Sheng, Z.; Oka, S.; Nakabeppu, Y. 8-Oxoguanine accumulation in mitochondrial DNA causes mitochondrial dysfunction and impairs neuritogenesis in cultured adult mouse cortical neurons under oxidative conditions. Sci Rep 2016, 6, 22086.
(55) Geiger, R.; Strasak, A.; Treml, B.; Gasser, K.; Kleinsasser, A.; Fischer, V.; Geiger, H.; Loeckinger, A.; Stein, J. I. Six-minute walk test in children and adolescents. J Pediatr 2007, 150, 395-399, 399 e391-392.
(56) Li, A. M.; Yin, J.; Au, J. T.; So, H. K.; Tsang, T.; Wong, E.; Fok, T. F.; Ng, P. C. Standard reference for the six-minute-walk test in healthy children aged 7 to 16 years. Am J Respir Crit Care Med 2007, 176, 174-180.
(57) Lammers, A. E.; Hislop, A. A.; Flynn, Y.; Haworth, S. G. The 6-minute walk test: normal values for children of 4-11 years of age. Arch Dis Child 2008, 93, 464-468.
(58) Bohannon, R. W.; Bubela, D. J.; Wang, Y. C.; Magasi, S. S.; Gershon, R. C. Six-Minute Walk Test Vs. Three-Minute Step Test for Measuring Functional Endurance. J Strength Cond Res 2015, 29, 3240-3244.
(59) Costedoat-Chalumeau, N.; Hulot, J. S.; Amoura, Z.; Delcourt, A.; Maisonobe, T.; Dorent, R.; Bonnet, N.; Sable, R.; Lechat, P.; Wechsler, B.; Piette, J. C. Cardiomyopathy related to antimalarial therapy with illustrative case report. Cardiology 2007, 107, 73-80.
(60) Chasset, F.; Frances, C. Current Concepts and Future Approaches in the Treatment of Cutaneous Lupus Erythematosus: A Comprehensive Review. Drugs 2019, 79, 1199-1215.
(61) Frangou, E.; Chrysanthopoulou, A.; Mitsios, A.; Kambas, K.; Arelaki, S.; Angelidou, I.; Arampatzioglou, A.; Gakiopoulou, H.; Bertsias, G. K.; Verginis, P.; Ritis, K.; Boumpas, D. T. REDD1/autophagy pathway promotes thromboinflammation and fibrosis in human systemic lupus erythematosus (SLE) through NETs decorated with tissue factor (TF) and interleukin-17A (IL-17A). Ann Rheum Dis 2019, 78, 238-248.
(62) An, N.; Chen, Y.; Wang, C.; Yang, C.; Wu, Z. H.; Xue, J.; Ye, L.; Wang, S.; Liu, H. F.; Pan, Q. Chloroquine Autophagic Inhibition Rebalances Th17/Treg-Mediated Immunity and Ameliorates Systemic Lupus Erythematosus. Cell Physiol Biochem 2017, 44, 412-422.
(63) Ratliff, N. B.; Estes, M. L.; Myles, J. L.; Shirey, E. K.; McMahon, J. T. Diagnosis of chloroquine cardiomyopathy by endomyocardial biopsy. N Engl J Med 1987, 316, 191-193.
(64) Piette, J. C.; Guillevin, L.; Chapelon, C.; Wechsler, B.; Bletry, O.; Godeau, P. Chloroquine cardiotoxicity. N Engl J Med 1987, 317, 710-711.
(65) Azimian, M.; Gultekin, S. H.; Hata, J. L.; Atkinson, J. B.; Ely, K. A.; Fuchs, H. A.; Mobley, B. C. Fatal antimalarial-induced cardiomyopathy: report of 2 cases. J Clin Rheumatol 2012, 18, 363-366.
(66) Roos, J. M.; Aubry, M. C.; Edwards, W. D. Chloroquine cardiotoxicity: clinicopathologic features in three patients and comparison with three patients with Fabry disease. Cardiovasc Pathol 2002, 11, 277-283.
(67) Macfarlane, D. E.; Manzel, L. Antagonism of immunostimulatory CpG-oligodeoxynucleotides by quinacrine, chloroquine, and structurally related compounds. J Immunol 1998, 160, 1122-1131.
(68) Hacker, H.; Mischak, H.; Miethke, T.; Liptay, S.; Schmid, R.; Sparwasser, T.; Heeg, K.; Lipford, G. B.; Wagner, H. CpG-DNA-specific activation of antigen-presenting cells requires stress kinase activity and is preceded by non-specific endocytosis and endosomal maturation. EMBO J 1998, 17, 6230-6240.
(69) Yi, A. K.; Tuetken, R.; Redford, T.; Waldschmidt, M.; Kirsch, J.; Krieg, A. M. CpG motifs in bacterial DNA activate leukocytes through the pH-dependent generation of reactive oxygen species. J Immunol 1998, 160, 4755-4761.
(70) French, J. K.; Hurst, N. P.; O'Donnell, M. L.; Betts, W. H. Uptake of chloroquine and hydroxychloroquine by human blood leucocytes in vitro: relation to cellular concentrations during antirheumatic therapy. Ann Rheum Dis 1987, 46, 42-45.
(71) Manzel, L.; Strekowski, L.; Ismail, F. M.; Smith, J. C.; Macfarlane, D. E. Antagonism of immunostimulatory CpG-oligodeoxynucleotides by 4-aminoquinolines and other weak bases: mechanistic studies. J Pharmacol Exp Ther 1999, 291, 1337-1347.
(72) Kuznik, A.; Bencina, M.; Svajger, U.; Jeras, M.; Rozman, B.; Jerala, R. Mechanism of endosomal TLR inhibition by antimalarial drugs and imidazoquinolines. J Immunol 2011, 186, 4794-4804.
(73) Lamphier, M.; Zheng, W.; Latz, E.; Spyvee, M.; Hansen, H.; Rose, J.; Genest, M.; Yang, H.; Shaffer, C.; Zhao, Y.; Shen, Y.; Liu, C.; Liu, D.; Mempel, T. R.; Rowbottom, C.; Chow, J.; Twine, N. C.; Yu, M.; Gusovsky, F.; Ishizaka, S. T. Novel small molecule inhibitors of TLR7 and TLR9: mechanism of action and efficacy in vivo. Mol Pharmacol 2014, 85, 429-440.
(74) Mauthe, M.; Orhon, I.; Rocchi, C.; Zhou, X.; Luhr, M.; Hijlkema, K. J.; Coppes, R. P.; Engedal, N.; Mari, M.; Reggiori, F. Chloroquine inhibits autophagic flux by decreasing autophagosome-lysosome fusion. Autophagy 2018, 14, 1435-1455.
(75) Cohen, S. N.; Yielding, K. L. Spectrophotometric Studies of the Interaction of Chloroquine with Deoxyribonucleic Acid. J Biol Chem 1965, 240, 3123-3131.
(76) Lin, J. Y.; Jing, R.; Lin, F.; Ge, W. Y.; Dai, H. J.; Pan, L. High Tidal Volume Induces Mitochondria Damage and Releases Mitochondrial DNA to Aggravate the Ventilator-Induced Lung Injury. Front Immunol 2018, 9, 1477.
(77) Spears, L. D.; Tran, A. V.; Qin, C. Y.; Hobbs, S. B.; Burns, C. A.; Royer, N. K.; Zhang, Z.; Ralston, L.; Fisher, J. S. Chloroquine increases phosphorylation of AMPK and Akt in myotubes. Heliyon 2016, 2, e00083.
(78) Jeong, H. Y.; Kang, J. M.; Jun, H. H.; Kim, D. J.; Park, S. H.; Sung, M. J.; Heo, J. H.; Yang, D. H.; Lee, S. H.; Lee, S. Y. Chloroquine and amodiaquine enhance AMPK phosphorylation and improve mitochondrial fragmentation in diabetic tubulopathy. Sci Rep 2018, 8, 8774.
(79) Deepalakshmi, P. D.; Parasakthy, K.; Shanthi, S.; Devaraj, N. S. Effect of chloroquine on rat liver mitochondria. Indian J Exp Biol 1994, 32, 797-799.
(80) Cerqueira, F. M.; Laurindo, F. R.; Kowaltowski, A. J. Mild mitochondrial uncoupling and calorie restriction increase fasting eNOS, akt and mitochondrial biogenesis. PLoS One 2011, 6, e18433.
(81) Dwyer, J. R.; Donkor, J.; Zhang, P.; Csaki, L. S.; Vergnes, L.; Lee, J. M.; Dewald, J.; Brindley, D. N.; Atti, E.; Tetradis, S.; Yoshinaga, Y.; De Jong, P. J.; Fong, L. G.; Young, S. G.; Reue, K. Mouse lipin-1 and lipin-2 cooperate to maintain glycerolipid homeostasis in liver and aging cerebellum. Proc Natl Acad Sci USA 2012.
(82) Jackowski, S. Coordination of membrane phospholipid synthesis with the cell cycle. J Biol Chem 1994, 269, 3858-3867.
(83) Yoshida, S.; Tamiya-Koizumi, K.; Kojima, K. Interaction of DNA polymerases with phospholipids. Biochim Biophys Acta 1989, 1007, 61-66.
(84) Shoji-Kawaguchi, M.; Izuta, S.; Tamiya-Koizumi, K.; Suzuki, M.; Yoshida, S. Selective inhibition of DNA polymerase epsilon by phosphatidylinositol. J Biochem 1995, 117, 1095-1099.
(85) Santos-Rosa, H.; Leung, J.; Grimsey, N.; Peak-Chew, S.; Siniossoglou, S. The yeast lipin Smp2 couples phospholipid biosynthesis to nuclear membrane growth. EMBO J 2005, 24, 1931-1941.
(86) Xagorari, A.; Chlichlia, K. Toll-like receptors and viruses: induction of innate antiviral immune responses. Open Microbiol J 2008, 2, 49-59.
(87) Barton, G. M. Viral recognition by Toll-like receptors. Semin Immunol 2007, 19, 33-40.
(88) Han, G. S.; Siniossoglou, S.; Carman, G. M. The cellular functions of the yeast lipin homolog PAH1p are dependent on its phosphatidate phosphatase activity. J Biol Chem 2007, 282, 37026-37035.
(89) Choi, H. S.; Su, W. M.; Han, G. S.; Plote, D.; Xu, Z.; Carman, G. M. Pho85p-Pho80p phosphorylation of yeast Pah1p phosphatidate phosphatase regulates its activity, location, abundance, and function in lipid metabolism. J Biol Chem 2012, 287, 11290-11301.
(90) Chuang, C.; Barajas, D.; Qin, J.; Nagy, P. D. Inactivation of the Host Lipin Gene Accelerates RNA Virus Replication through Viral Exploitation of the Expanded Endoplasmic Reticulum Membrane. PLoS Pathog 2014, 10, e1003944.
(91) Belov, G. A. Modulation of lipid synthesis and trafficking pathways by picornaviruses. Curr Opin Virol 2014, 9C, 19-23.
(92) den Boon, J. A.; Diaz, A.; Ahlquist, P. Cytoplasmic viral replication complexes. Cell Host Microbe 2010, 8, 77-85.
(93) Greseth, M. D.; Traktman, P. De novo fatty acid biosynthesis contributes significantly to establishment of a bioenergetically favorable environment for vaccinia virus infection. PLoS Pathog 2014, 10, e1004021.
(94) Fodili, F.; van Bommel, E. F. Severe rhabdomyolysis and acute renal failure following recent Coxsackie B virus infection. Neth J Med 2003, 61, 177-179.
(95) Perrin-Cocon, L.; Aublin-Gex, A.; Sestito, S. E.; Shirey, K. A.; Patel, M. C.; Andre, P.; Blanco, J. C.; Vogel, S. N.; Peri, F.; Lotteau, V. TLR4 antagonist FP7 inhibits LPS-induced cytokine production and glycolytic reprogramming in dendritic cells, and protects mice from lethal influenza infection. Sci Rep 2017, 7, 40791.
(96) Torun, S. H.; Karakilic, E.; Akturk, H.; Sutcu, M.; Uysalol, M.; Ciplak, M.; Badur, S.; Salman, N.; Somer, A. Influenza in the pediatric population in Istanbul: a one center experience 2009-2014. Epidemiol Mikrobiol Imunol 2016, 65, 46-50.
(97) Callado, R. B.; Carneiro, T. G.; Parahyba, C. C.; Lima Nde, A.; da Silva Junior, G. B.; Daher Ede, F. Rhabdomyolysis secondary to influenza A H1N1 vaccine resulting in acute kidney injury. Travel Med Infect Dis 2013, 11, 130-133.

1. A method of treating a patient suffering from Lipin-1 deficiency, the method comprising administering an amount of hydroxychloroquine (HCQ) sufficient to elicit a plasma level that does not exceeds 700 ng/ml.

2. The method of claim 1 wherein the HCQ is administered to the patient so as to elicit a plasma level that ranges from 100 ng/ml to 700 ng/ml.
